(19) United States
(12) Patent Application Publication
(10) Pub. No.: US 2014/0246624 A1
(43) Pub. Date: Sep. 4, 2014

(54) MONOCHROMATIC DOT ENSEMBLES

(71) Applicant: RareCyte, Inc., Seattle, WA (US)

(72) Inventors: Ronald Seubert, Sammamish, WA (US); Paul Spatafore, Bothell, WA (US); Joshua Nordberg, Bainbridge Island, WA (US)

(73) Assignee: RareCyte, Inc., Seattle, WA (US)

(21) Appl. No.: 14/193,707

(22) Filed: Feb. 28, 2014

Related U.S. Application Data
(60) Provisional application No. 61/771,697, filed on Mar. 1, 2013.

(51) Int. Cl. C09K 11/88; C09K 11/70; B07C 5/34; C09K 11/74; C09K 11/59; C09K 11/65; G01N 33/58; C09K 11/56

(52) U.S. Cl. CPC C09K 11/883; G01N 33/588; C09K 11/70; C09K 11/881; C09K 11/565; C09K 11/7492; C09K 11/59; C09K 11/65; B07C 5/34
USPC 252/301.4 R; 423/299; 423/509; 423/566.1; 423/348; 423/449.1; 209/576

(57) ABSTRACT

This disclosure is directed to systems and methods for sorting a native aggregate, such as a fluorescent nanoparticle aggregate, which includes multiple objects, some of which have different characteristics, into lower level ensembles, such as monochromatic nanoparticle ensembles. In one aspect, the system includes two detectors, one of which accepts all emitted wavelengths and another one which is preceded by a filter to permit transmission of a specific wavelength or range of wavelengths. In another aspect, the system includes multiple detectors, each detector configured to detect a given wavelength or range of wavelengths, such that no two detectors have overlapping wavelengths or ranges. In yet another aspect, the system includes an optical regulator in front of a detector. This disclosure is also directed to systems and methods for multiplexing and analyzing a target analyte using the monochromatic nanoparticle ensembles.

### CROSS-REFERENCE TO A RELATED APPLICATION

This application claims the benefit of Provisional Application No. 61/771,697, filed Mar. 1, 2013.

### TECHNICAL FIELD

This invention relates generally to solution separation and, in particular, to systems and methods for multiplexing and for separating native aggregates into narrower ensembles.

### BACKGROUND

Whole blood is a suspension of particles (e.g., red blood cells and white blood cells) in a proteinaceous liquid (plasma). Whole blood is routinely examined for the presence of abnormal organisms or cells, such as fetal cells, nucleic acids, parasites, microorganisms, and inflammatory cells. Recently blood has been examined for the presence of cancer cells. Blood is typically analyzed by smearing a sample on a slide and is stained and visually studied usually by bright field microscopy, and then, when needed, by immunologic stains and/or other molecular techniques. Visual detection of cancer cells or foreign bodies in blood is dependent on the efficient labeling of targets with a fluorescent tag and the microscopic detection of that tag, or on morphological characteristics of cancer cells or foreign bodies.

Whole blood samples can also be collected to detect a variety of different viruses, for example, HIV, cytomegalovirus, hepatitis C virus, and Epstein-Barr virus. In some cases, practitioners, researchers, and those working with various samples use dyes and fluorescent proteins to detect these viruses. Unfortunately, these dyes and fluorescent proteins have a number of disadvantages which may render their observation inconclusive. The dyes and fluorescent proteins are not very bright, have wide emission spectra, and suffer from photobleaching.

### BRIEF DESCRIPTION OF THE DRAWINGS

FIG. 1A shows an example quantum dot aggregate including example quantum dots.

FIG. 1B shows a target analyte conjugated with the example quantum dots of the example quantum dot aggregate.

FIG. 1C shows the spectral emission of an example quantum dot of the example quantum dots.

FIG. 2 shows a monochromatic dot ensemble.

FIGS. 3A-3G show example analyzers.

FIG. 4A-4B show an example imager.

FIG. 4C-4E show example imagers.

FIG. 4F shows an example imager.

FIG. 5A shows a target analyte labeled with multiple quantum dots from multiple monochromatic dot ensembles.

FIG. 5B shows a DNA origami tagged with quantum dots from multiple monochromatic dot ensembles.

### DETAILED DESCRIPTION

This disclosure is directed to systems and methods for sorting a native aggregate, such as a fluorescent nanoparticle aggregate, which includes multiple objects, some of which have different characteristics, into lower level ensembles, such as monochromatic nanoparticle ensembles. In one aspect, the system includes two detectors, one of which accepts all emitted wavelengths and another one which is preceded by a filter to permit transmission of a specific wavelength or range of wavelengths. In another aspect, the system includes multiple detectors, each detector configured to detect a given wavelength or range of wavelengths, such that no two detectors have overlapping wavelengths or ranges. In yet another aspect, the system includes an optical regulator in front of a detector. This disclosure is also directed to systems and methods for multiplexing and analyzing a target analyte using the monochromatic nanoparticle ensembles.

In the following description, the term “light” is not intended to be limited to describing electromagnetic radiation in the visible portion of the electromagnetic spectrum, but is also intended to describe radiation in the ultraviolet and infrared portions of the electromagnetic spectrum.

For the sake of convenience, the methods and systems are described with reference to an example light source of quantum dots and quantum dot aggregates. But the methods and systems described below are not intended to be so limited in their scope of application. The methods and systems, in practice, can be used with any kind of light-emitting or fluorescent nanoparticle and are not intended to be limited to quantum dots and quantum dot aggregates. For example, light-emitting or fluorescent nanoparticles may include, but are not limited to, nanometer-sized detection probes, nanodiamonds, Cornell dots (fluorescent dye encapsulated in a silica shell), DNA barcodes, and the like.

It should be further noted that the emission width, range, spectrum, or deviation of the fluorescent nanoparticles is represented by full width at half max (“FWHM”). The emission width, range, spectrum, or deviation may occur at any appropriate temperature, including, but not limited to, at or below freezing, between freezing and room temperature, at approximately room temperature, above room temperature, or the like.

### Quantum Dots

Quantum dots are small nanometer sized particles with multiple unique properties. The quantum dots are made from periodic groups II-VI, III-V, or IV-VI materials including, but not limited to cadmium selenide, cadmium sulfide, zinc sulfide, zinc telluride, indium arsenide, and indium phosphide. Quantum dots, as referenced herein, shall also include luminescent silicon and carbon-based nanoparticles.

The primary advantages of quantum dots include: archival capability, since there is little or no noticeable bleaching or fading after years; quantitative fluorescence, providing a characteristic, repeatable emission for a given amount of incident photon flux; extremely high photo efficiencies, approaching 90%; emission lifetimes that can be orders of magnitude longer than organic fluorophores; ability to absorb high photon flux; external surface capable of conjugating to molecular markers, such as antibodies; narrow emission spectra for individual quantum dots; and small sizes of 1-100 nm.

Quantum dots are important markers for research, discovery and diagnostic applications. Archival stability is desirable for slide specimens, such as biopsies for pathological examination, which may need multiple examinations and re-examination over times ranging from days to years. Minimal photobleaching, if any, permits the sample to be stored for an extended amount of time for multiple examinations as needed or required. The various attributes of quantum dots, such as bright emissions, also make it possible for in-vivo imaging of model organisms such as mice. Quantum dots may also be used to provide controlled light sources for scientific applications. It should be further noted that as the number of quantum dots in a sample increases, the brightness of the light emission increases. The brightness of the light emission is therefore proportional to the number of quantum dots present.

A quantum dot aggregate is typically created with various sizes of quantum dots within one manufacturing sample. Quantum dots are typically manufactured by “growing” a core substrate in solution or by performing laser ablation or electric arc discharge of certain materials. The distribution of quantum dot sizes and the resulting emission spectra (since the emission spectrum can be dependent on the size of the quantum dot) fall into a standard bell curve. The fluorescent property of the quantum dot may be based on the size, on the number of layers or coatings, or an attached particle, depending on the base from which the quantum dot is derived or other attributes. The quantum emission of light is very important. An individual quantum dot can emit monochromatic light, with an emission width only a few nanometers in width, even as narrow as 1 nm (at approximately 5 Kelvin). Small changes in physical properties of quantum dots, such as size, may shift the emission wavelength slightly, thereby providing individual quantum dots with different emission colors.

The broad emission spectrum of a quantum dot aggregate reduces the functionality of quantum dots for some applications. For example, in imaging applications for research and diagnostics it is desirable to simultaneously detect and observe as many objects as practical. The broad emission of ensemble quantum dots causes difficulties in separating the overlapping spectra from one quantum dot aggregate from the spectra of an adjacent quantum dot aggregate. For example, even with a combination of interference filters that have exceptionally narrow band passes, differentiating between quantum dots in a 525 nm ensemble from those in a 545 nm ensemble would be difficult due to the overlap in each ensemble's broad emission curve. Complex methods have been developed, usually involving spectral unmixing in an attempt to resolve this problem but these methods are time consuming, and may even provide an incorrect result. As assays become more sensitive and attempt to identify lower concentrations of analyte, fewer quantum dots will be present in the sample. The underlying ensemble distribution may no longer accurately represent this sparse quantum dot distribution. Indeed, as the number of quantum dots is reduced it may be difficult to determine whether the emission at 545 nm is from the ensemble of 525 nm, 545 nm, or 565 nm.

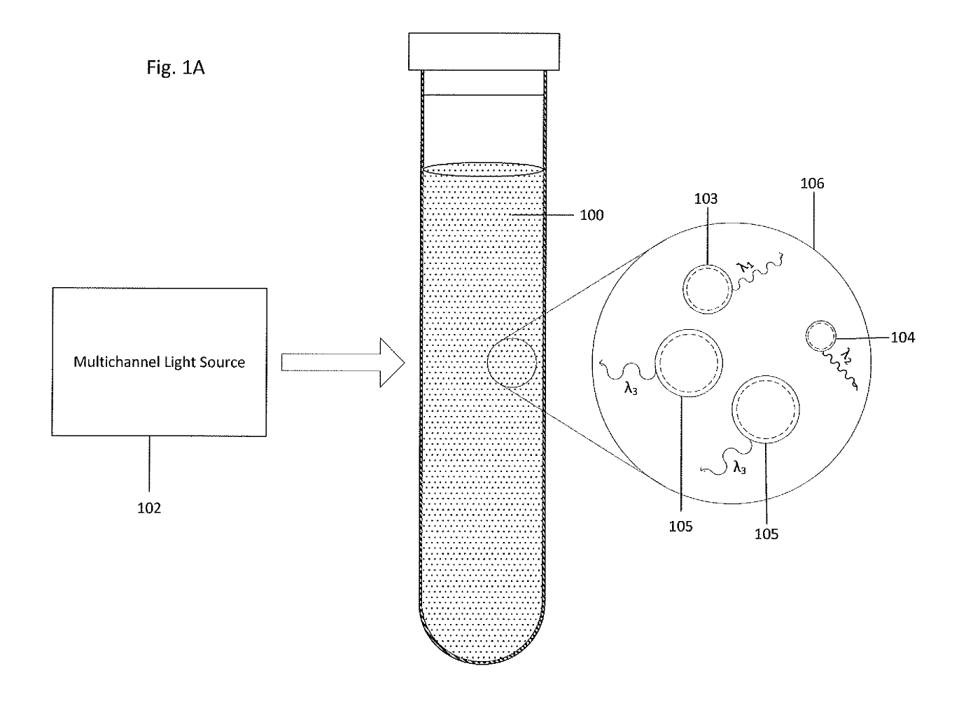

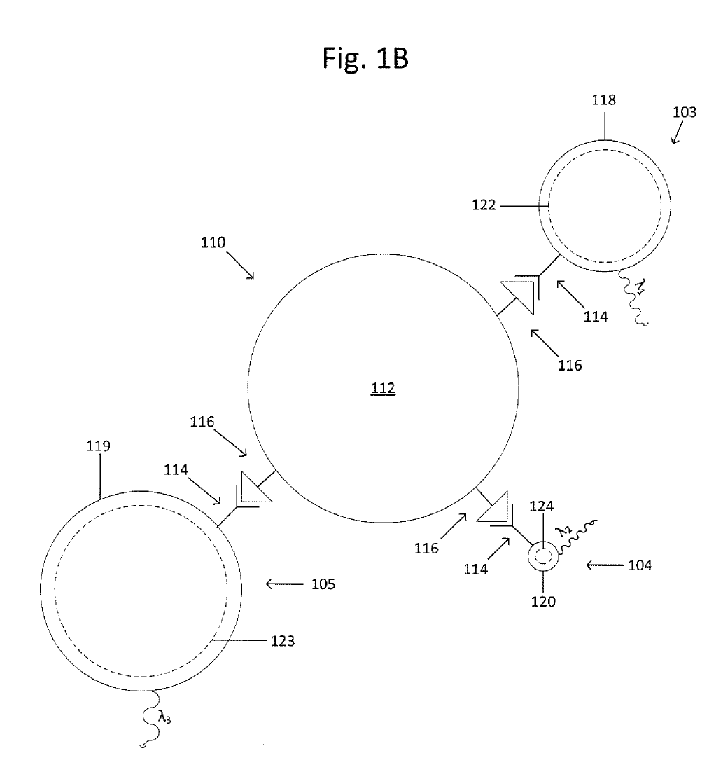

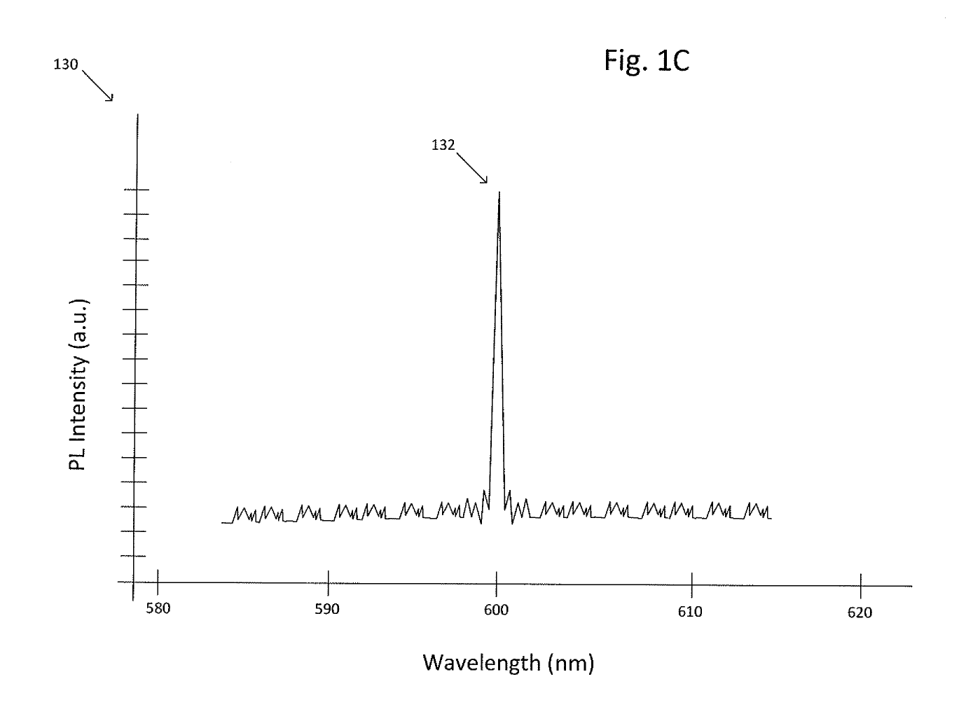

FIG. 1A shows a quantum dot aggregate 100. Snapshot 106 shows quantum dots 103-105 and respective emissions wavelengths λ₁-λ₃ of an emission light that the quantum dots 103-105 emit when excited by a light produced by a light source 102. FIG. 1B shows the quantum dot aggregate 100 conjugated to a target analyte 112. In the quantum dot aggregate 100, shells 118-120 of the quantum dots 103-105 are made of the same material, though the shells 118-120 are different sizes. In the quantum dot aggregate 100, cores 122-124 of the quantum dots 103-105 are made of the same material, though the cores 122-124 are different sizes. The quantum dots 103-105 are bound to an antibody 114 to conjugate to a protein, such as surface protein 116, of the target analyte 112. Since the quantum dot aggregate 100 includes quantum dots 103-105 of varying sizes, and the emission light is related to quantum dot size, the emission light of the quantum dot aggregate 100 is not monochromatic. FIG. 1C shows light-emission characteristics 130 of an individual quantum dot. The quantum dot emits exquisitely narrowband light with an emission peak 132 as low as 1 nm in width.

### Monochromatic Dot Ensembles

A “monochromatic dot ensemble” (“MDot ensemble”) is an ensemble with a spectral range that is less than the spectral range of a native aggregate. The native aggregate may be a quantum dot aggregate or any higher level aggregate which includes multiple objects, some of which have different characteristics, such that it may be desirous to sort, develop, or separate those objects based on their differing characteristics. The MDot ensemble spectral range may be from 100 nanometers down to 1 or 2 nanometers. Sorting quantum dot aggregates into MDot ensembles is accomplished by analyzing the emission wavelength of each quantum dot, and then directing the quantum dot into an appropriate MDot ensemble, such that each quantum dot within a given MDot ensemble has an emission wavelength which deviates from a specified wavelength by a pre-determined amount. The pre-determined deviation may be 0 to 500 nm from the specified wavelength (i.e. ±0-500 nm from specified wavelength), including deviations of 50 nm, 10 nm, 5 nm, 1 nm. The monochromatic dot ensemble may have an emission spectra that is less than or equal to one-half of the emission spectra of the native aggregate from which the monochromatic dots were sorted, separated, or developed. The monochromatic dot ensemble may have an emission spectra that is less than or equal to one-third, one-quarter, one-tenth, one-hundredth or the like of the emission spectra of the native aggregate from which the monochromatic dots were sorted, separated, or developed. Sorting by emission wavelength maintains a consistently narrow spectrum within the MDot ensemble, whereas size sorting does not.

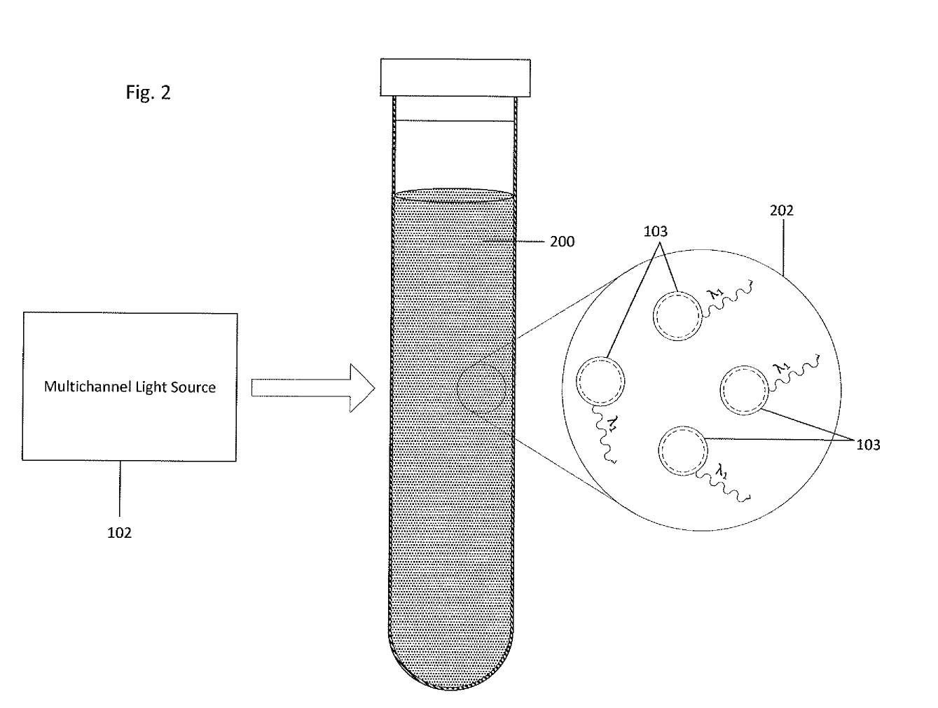

FIG. 2 shows a MDot ensemble 200. The MDot ensemble 200 includes quantum dots 103 having the same emission spectra which have been separated from the quantum dot aggregate 100 as depicted in FIG. 1A. The MDot ensemble 200 may include a single quantum dot or many quantum dots. Snapshot 202 shows quantum dots 103 and respective emissions with wavelengths λ₁ that the quantum dots 103 emit when excited by the light produced by the light source 102. The quantum dots 103 therefore have the same emissions with wavelength λ₁.

### I. Sorting and Detecting

The systems disclosed are capable of separating a native aggregate into narrower ensembles and detecting a multiplexed analyte. When sorting, the systems separate a quantum dot aggregate into MDot ensembles. When detecting, the systems analyze an analyte for the presence of quantum dots from multiple MDot ensembles.

For the sake of convenience, the methods and systems are described with reference to a system using hydrodynamic focusing, such as a fluorescence activated cell sorter (“FACS”), or using an imaging device, such as a fluorescent microscope. But the methods and systems described below are not intended to be so limited in their scope of application. For example, a quantum dot aggregate may be sorted based on the mass or the size of the quantum dots, such as by performing ultracentrifugation, gel electrophoresis, or high pressure liquid chromatography. The methods and systems, in practice, can be used with any kind of analysis or imaging device, such as a microfluidic chip. For example, a sample may by analyzed using digital microfluidics. In digital microfluidics, the sample is introduced onto a chip and is able to be controlled to perform a multitude of functions with the use of electrodes. An analyte may be analyzed for emission light or lights and a quantum dot aggregate may be separated into MDot ensembles. Furthermore, when using hydrodynamic focusing or any fluidics, the flow rate of the sample may be reduced so as to increase exposure time to both a light source and a detector. Furthermore, the system, such as the FAGS or microfluidic chip, may include a mirrored surface or many mirrored surfaces to reflect the emission light to a detector, thereby increasing the amount of emission light that is captured by the detector. Furthermore, a light source and a detector may be elongated, so as to excite and detect over a greater distance, thereby allowing for increased detection time.

Identifying multiple emissions of MDots may be accomplished by several methods, such as interference filters or spectral-based systems such as an acousto-optical tunable filter (“AOTF”), filter wheel, digital microfluidics, or tunable LCD system. For example, a MDot ensemble is provided with a distribution less than or equal to 5 nm. In this instance, 5 nm bandpass filters on 10 nm centers may be used to detect up to 41 biological markers in the visible (375-775 nm) spectrum, though any emission is contemplated within the spectrum ranging from about 390 nm to about 1000 nm or greater, limited only by the emission characteristics of the quantum dot aggregate. Using narrower filters could increase the density to over 100 markers in a single sample. Using 1 nm bandpass filters may permit the operator to select even more biological markers from a given sample.

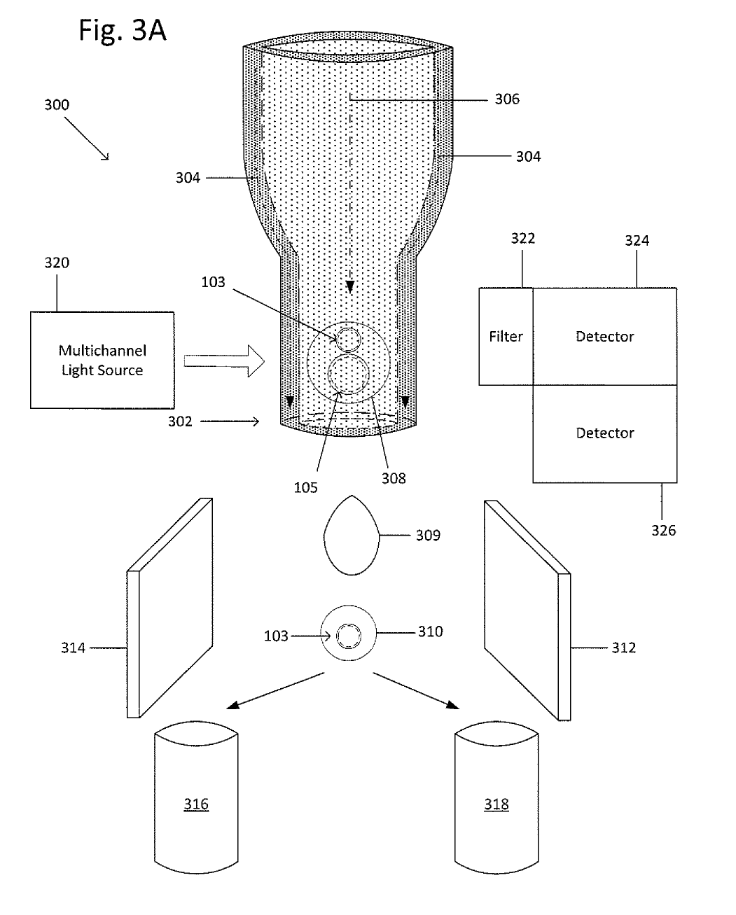

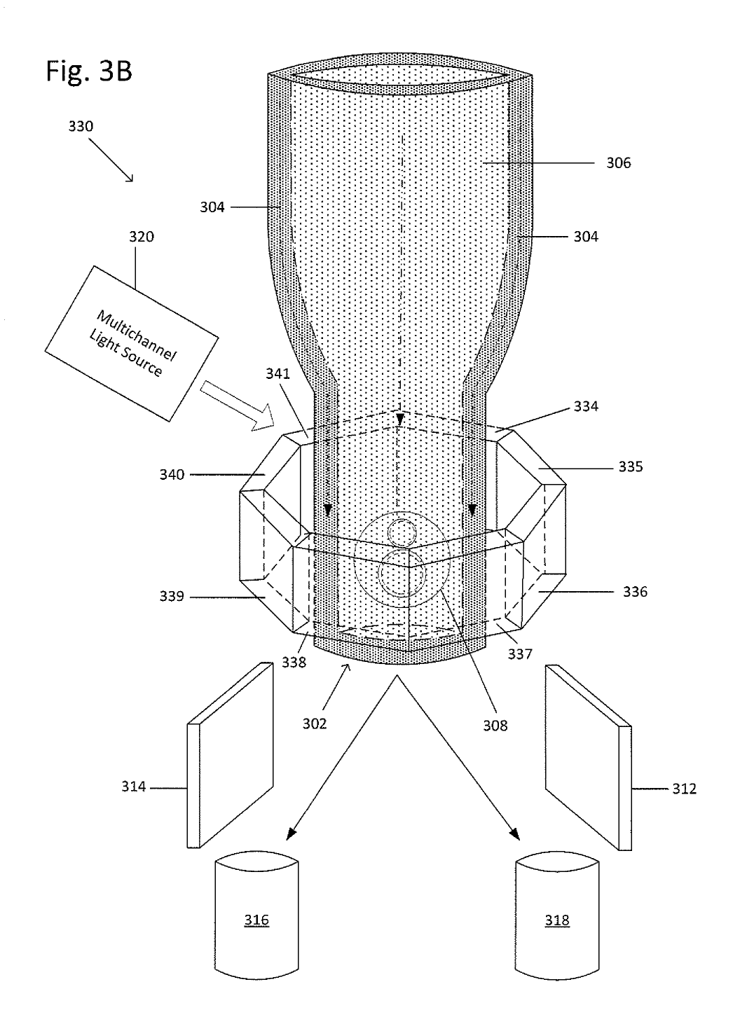

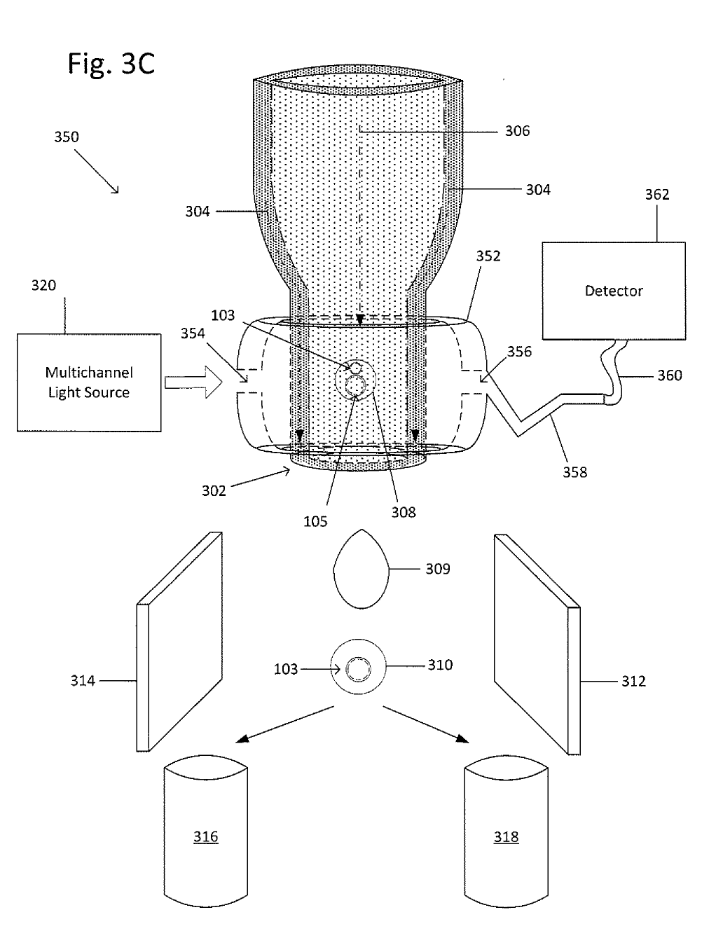

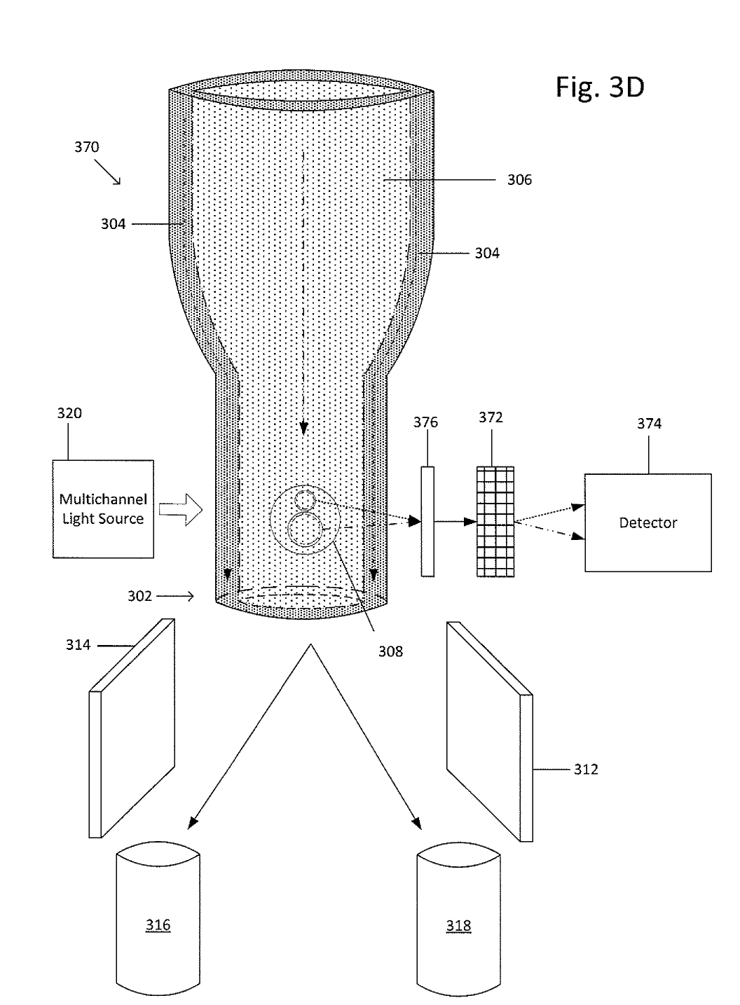

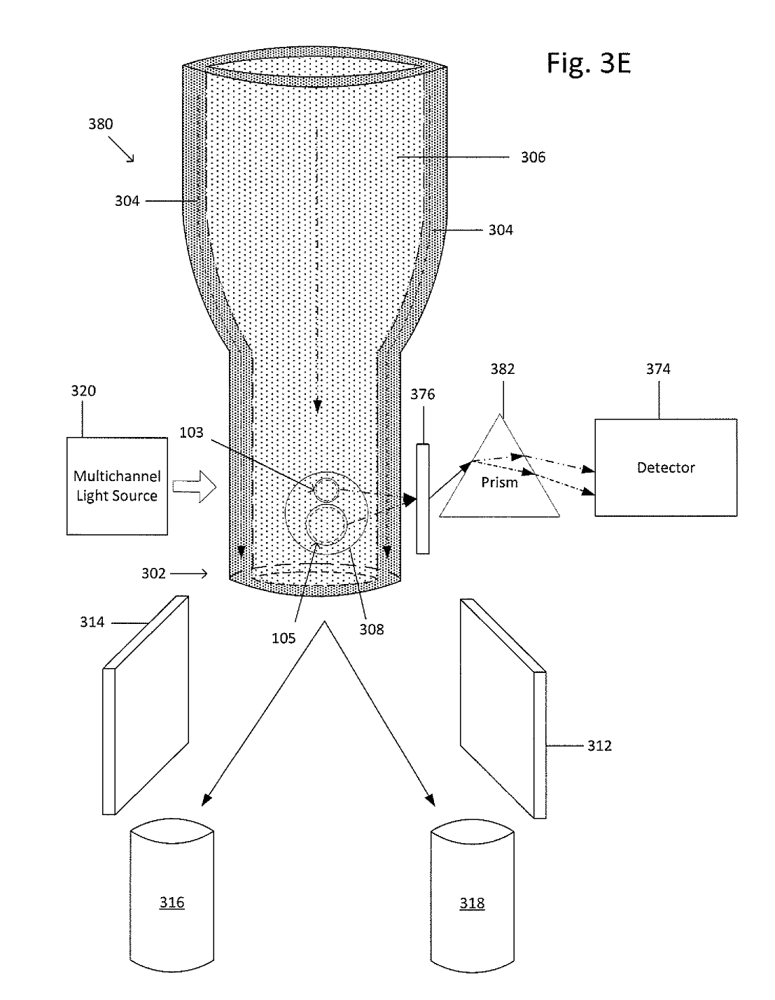

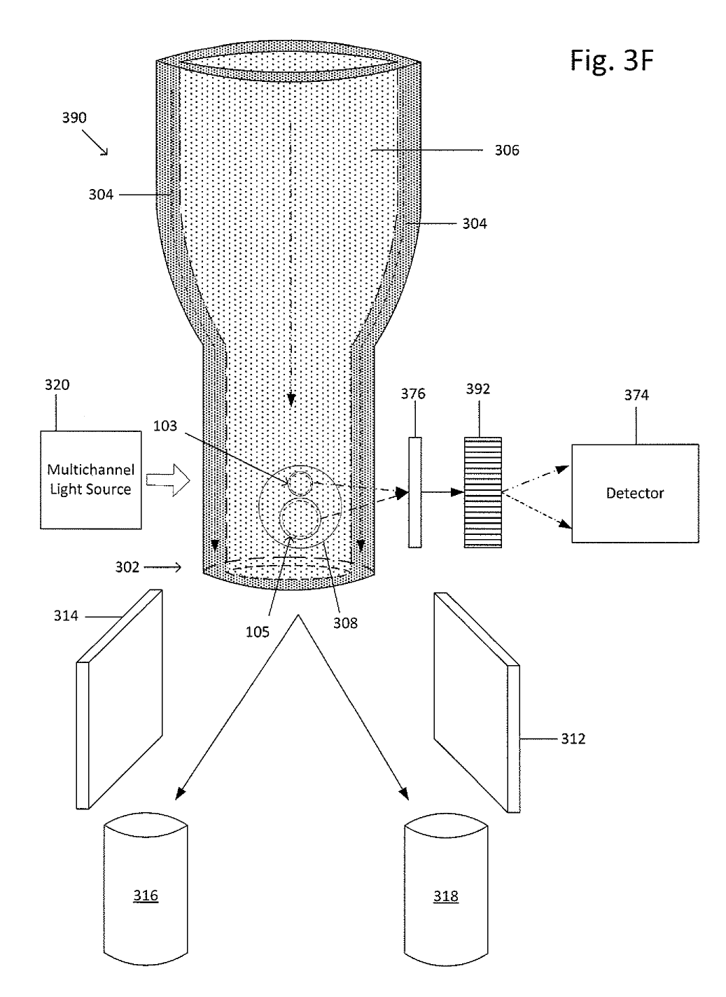

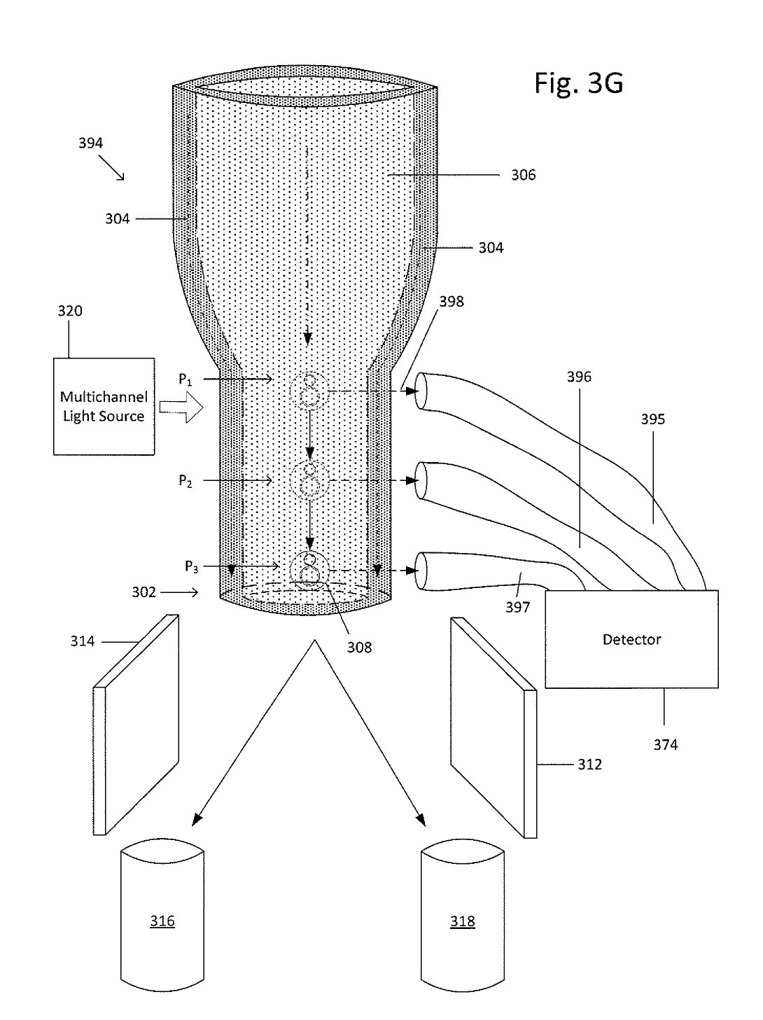

FIG. 3A shows an analyzer 300. A fluid 306 is first formed into a single dot stream with the aid of a sheath fluid 304 through a nozzle 302 using hydrodynamic focusing. The fluid 306 may be a solution, a suspension, such as a biological fluid suspected of including a target analyte or a quantum dot aggregate, or the like. In hydrodynamic focusing, the fluid 306 is constricted by the different flow rate of the sheath fluid 304. Hydrodynamic focusing maintains the single dot stream for use within the system for the detection and analysis of a particle based on the light emission frequency of corresponding quantum dots. A light source 320 bombards the single dot stream fluid with excitation light. The single dot stream fluid may emit an emission light when a quantum dot is excited by the excitation light at given point in the nozzle 302; the single dot stream fluid may emit multiple emission lights when multiple quantum dots are excited simultaneously by the excitation light at the given point in the nozzle 302; or, the single dot stream fluid may not emit any emission lights when no quantum dots are present at the given point in the nozzle 302. The fluid is broken up into droplets at the end of the nozzle 302, each droplet is charged, and the droplet is then diverted to a vessel via charged deflection plates 312 and 314 based on the detected emission, detected emissions, or absence of any emission.

A first filter 322 may be placed in front of a first detector 324. The first filter 322 may be a narrow bandpass filter, thereby permitting an emission that occurs within the narrow band to pass through to the first detector 324, such that the first detector 324 detects a first, narrow emission. A second detector 326 detects all emissions. A second filter (not shown) may be placed in front of the second detector 326 to permit passage of all emissions while blocking the passage of the light produced by the light source. The emissions detected by the first and second detectors 324 and 326 are compared. When the emissions are the same, the droplet, such as droplet 310, may be diverted into a first vessel 316; when the emissions are different, such as in droplet 308, or no emissions are detected, such as in droplet 309, by the first detector 322, the droplet, may be diverted into a second vessel 318. The accumulation then present in the second vessel 318 may then be re-processed for further separation. Alternatively, when there are no emissions detected by the first detector 322, the droplet, such as droplet 309, may be diverted into a third vessel (not shown), while the droplet 308 may be diverted to the second vessel 318.

Alternatively, the analyzer 300 may also include more than 2 vessels into which the quantum dots are separated. Each vessel may have a different frequency range and the quantum dots can be diverted into the respective vessels. A quantum dot aggregate can therefore be broken up into multiple MDot ensembles without having to re-process and re-separate each vessel.

Alternatively, the first and second detectors 324 and 326 and the first filter 322 may be integrated into a fluorescent microscope, including a scanning fluorescent microscope. The fluid 306 may be analyzed on a microscope slide or in a vessel, such as a tube.

The filter may be a bandpass filter, an interference filter, a dichroic filter, or a tunable filter, such as an AOTF. The tunable filter allows for selection and transmission of a single wavelength of light. Any size bandpass filters are contemplated, including 1 nm, 5 nm, and 10 nm bandpass filters. The bandpass filters may also be used during multiplexed imaging and detection. When multiple bandpass filters are used, the bandpass filters may be arranged within a filter wheel or among different detectors.

Another method for separation of a quantum dot aggregate involves the use of successive detectors and deflectors. By separating the quantum dot aggregate at successive levels, the accumulations at each respective level may be separated further and further into narrower and narrower accumulations the initial quantum dot aggregate is separated into appropriate MDot ensembles. The first level will be “coarser” in nature, thereby having first-level containers with wider emission frequency ranges. The subsequent levels will be “finer” in nature, thereby having successive containers with narrower emission frequency ranges. The process occurs in successive levels until the quantum dot aggregate is broken up into containers having appropriate MDot ensembles. The MDot ensembles may include emission ranges from 0 nm to 100 nm, wherein the emission range is the difference between the wavelength of an emission light of the longest wavelength emitted by a quantum dot in the MDot ensemble and the wavelength of an emission light of the shortest wavelength emitted by another quantum dot in the MDot ensemble.

FIG. 3B shows an analyzer 330. The analyzer 330 is similar to the analyzer 300 discussed above, except that analyzer 330 includes detectors 334-341, such that each detector is capable of detecting a specific range of wavelengths or an individual wavelength that does not overlap with any other wavelength or wavelengths detectable by another detector. The detectors 334-341 may be formed in any appropriate configuration, such a ring, in a line, in a stack, staggered, or the like, such that each detector is capable of receiving emission light. It should be further noted that any number of detectors equal to or greater than 2 are contemplated. Filters (not shown), such as narrow bandpass filters or tunable filters, may be placed in front of each one of the detectors 334-341. Each one of the filters permit passage, and subsequent detection, of a specific wavelength. No two filters permit passage of overlapping or equal wavelengths. Each one of the respective filters corresponds to one of the detectors 334-341, such that there is an equal number of detectors and respective filters. The respective filters are capable of preventing the light emitted by the light source 320 from passing to each one of the detectors 334-341. The number of detectors is only limited by the number of MDot ensembles into which the quantum dot aggregate is to be sorted or the number of MDot ensembles used.

Each one of the detectors 334-341 is configured to detect a single emission wavelength from a fluid at a given point, such that none of the detector ranges overlap with another detector range. The fluid is broken up into droplets, such as droplet 308, at the end of the nozzle 302, each droplet is charged, and the droplet is then diverted to a vessel via charged deflection plates 312 and 314 based on the detected emission, detected emissions, or absence of any emission. To sort, for example, a droplet including a single emission wavelength may be diverted into a first container, whereas another droplet including multiple emission wavelengths may be diverted into a second container (and subsequently re-processed to further sort). For example, an analyte may be conjugated with quantum dots from different MDot ensembles. A first detector detects a first wavelength emission; a second detector detects a second wavelength emission; and so on. Based on the detected wavelengths, the analyte is included in a droplet, the droplet is charged, and the droplet is then diverted to a vessel via charged deflection plates based on the detected emission or emissions. An analyte emitting 5 wavelengths may be diverted into a vessel for collecting circulating tumor cells, whereas an analyte emitting 3 wavelengths may be diverted into a vessel for collecting white blood cells.

Alternatively, a series of filters, such as bandpass filters may be used to reduce the number of detectors. A first line of filters include keying filters, such that each keying filter is linked to a series of subsequent filters, such as dichroic filters, based on the wavelength of the signal. For example, keying filter #1 is linked to Filter A₁, Filter B₁, Filter C₁, and Filter D₁. When the signal has a wavelength appropriate for keying filter #1, the signal is also processed by Filters A₁-D₁. Keying filter #2 is linked to Filter A₂, Filter B₂, Filter C₂, and Filter D₂, and so on.

FIG. 3C shows an analyzer 350. The analyzer 350 is similar to the analyzer 300, except that the analyzer 350 includes an integrating sphere 352. The integrating sphere 352 acts as a light collector. The emission lights from all facets of the quantum dots 103 and 105 are diffused within the integrating sphere 352 and undergo multiple reflections. The different light diffusions converge at a singular point to increase detection sensitivity. The integrating sphere 352 includes an excitation port 354—to permit the excitation light from the multichannel light source 320 to pass through—and an emission port 356—to permit the collected light to exit the integrating sphere 352. The collected light may be detected by a detector 362; or, the collected light may be collected by a baffle 358, passed to a fiber optic cable 360, and then to the detector 362. Alternatively, the baffle 358 or the emission port 356 may be connected to many fiber optic cables, such as two or more. Each fiber optic cable receives emission lights having different wavelengths. The fiber optic cables then transmit the emission lights to the detector 362 or many detectors. When many detectors are used, each fiber optic cable transmits the emission light to a respective detector. Additionally, the analyzer 350 may include an amplifier, such as an optical amplifier, to amplify the emission light for detection.

An analyzer may include an optical regulator, such a photomask, a prism, a grating, a grating prism, or the like, to manipulate emission lights prior to being detected by a detector. FIG. 3D shows an analyzer 370. The analyzer 370 is similar to the analyzer 300 discussed above, except that the analyzer 370 includes a detector 374 and a photomask 372. The photomask 372 causes a singular signal to appear multiple times on the detector 374 in an array of X×Y, where X and Y are integers greater than or equal to 1. The photomask 372 may include a lens array of A×B, such that A and B are integers greater than or equal to 1, to project each copied image onto the detector 374. A filter array (not shown) may be used intermediate of the photomask 372 and the detector 374. The filter array (not shown) includes M×N filters, where M and N are integers greater than or equal to 1 and where there are an equal number of filters in the filter array and signals produced by photomask 372. Each filter of the filter array (not shown) may be a narrow bandpass filter. Each filter of the filter array (not shown) corresponds to a signal produced by the photomask 372. The filter array (not shown) is configured to separately process each copied signal for a wavelength specific to the individual copied signal. The filter array (not shown) may further be configured such that none of the filter ranges overlap with another filter range (i.e. no two filters overlap in wavelength spectra permitted to pass through). The detector 374, based on the number of copies produced by the photomask 372, is configured to determine individual emission wavelengths within each individual image. For example, a photomask produces a 5×5 array, thereby resulting in 25 images. A first image is analyzed for a 480 nm wavelength; a second image is analyzed for a 560 nm wavelength; and so on. The detector 374 is therefore capable of analyzing and detecting multiple wavelengths using a single image, though the image is copied multiple times. The analyzer 370 may also include an objective 376. The objective 376 collects all of the emission lights and focuses the emission lights on a singular point, such as on the photomask 372. The detector 374 may be a single detector or a detector array, such that each detector of the detector array receives a corresponding signal and is configured to only detect a specific wavelength range, such that no two wavelength ranges from different detectors in the detector array overlap. The detector array (not shown) includes O×P detectors, where O and P are integers greater than or equal to 1.

Alternatively, a tunable filter or a filter wheel including multiple bandpass filters may be used. Multiple images may be taken, whereby each image only contains a singular wavelength, and each image is then analyzed individually. For example, a first bandpass filter on the filter wheel may allow light having a wavelength between 475 nm and 485 nm to pass. The 480 nm wavelength will pass and an image will be taken. The bandpass filter, being either tunable or having a multiple filters, each filter having a distinct range, may then permit the passage of light having a wavelength of 550 nm to 560 nm. An image may then be taken. Each image, having distinct wavelengths displayed, may be analyzed. A secondary filter (not shown) may also be used to permit the passage of emission light or lights, while inhibiting the passage of scatter from the light source 320.

FIG. 3E shows an analyzer 380. The analyzer 380 is similar to the analyzer 370 discussed above, except that the analyzer 380 includes a prism 382. The prism 382 is an object which refracts light. As light enters a new medium, the light changes speed and refracts, thereby entering the medium at a different angle. The individual light colors making up the light refract are refracted differently, thereby leaving the prism 382 at different angles. By placing the prism 382 in front of the detector 374, the components of the original signal can be dispersed across the detector 374 due to the different angles at which the components are refracted by the prism 382. Breaking up the original signal into its components allows for the detection of the individual components, as the components do not mix with, dilute, weaken, or attenuate each other. The analyzer 380 may also include the objective 376. The objective 376 collects all of the emission lights and focuses the emission lights on a singular point, such as on the prism 382. Furthermore, many prisms, such as two or more, may be used in succession so to cause greater dispersion over the detector 374, as each successive prism increases the dispersion of the signal. The prism may be any appropriate shape, including, but not limited to, triangular or polyhedral. The prisms may be placed perpendicular to one another so as to spread the light in a planar manner.

FIG. 3F shows an analyzer 390. The analyzer 390 is similar to the analyzer 380 discussed above, except that the analyzer 390 includes a grating 392. The grating 392, such as a diffraction grating, is an object which splits and diffracts light based on the light components. Similar to the prism 382 discussed above, by placing the grating 392 in front of the detector 374, the components of the original signal can be dispersed across the detector 374 due to the different angles at which the components are split and diffracted by the grating 392. Breaking up the original signal into its components allows for the detection of the individual components, as the components do not mix with, dilute, weaken, or attenuate each other. The analyzer 390 may also include the objective 376. The objective 376 collects all of the emission lights and focuses the emission lights on a singular point, such as on the grating 392. Furthermore, many gratings, such as two or more, may be used in succession so to cause greater dispersion over the detector 374, as each successive grating increases the dispersion of the signal.

Alternatively, a grating prism may be used. The grating prism is a combination of a prism and a grating, such that a chosen wavelength is permitted to pass straight through. The grating prism may act like a filter by passing a chosen wavelength. However, the grating prism does not reject other wavelengths, if present. By diffracting and/or refracting the non-desired wavelengths, the grating prism may allow for subsequent processing of the non-desired wavelengths.

FIG. 3G shows an analyzer 394. The analyzer 394 is similar to the analyzer 300, except that the analyzer 394 includes fiber optic cables 395-398. The fiber optic cables 395-397 may be set at different positions or heights to capture an emission light 398 from the droplet 308 as the droplet 308 moves from positions or heights P₁ to P₂ to P₃. Each fiber optic cable at each position transmits the emission light 398 to the detector 374. Fiber optic cables 395-397 permit the emission light 398 to be captured, and therefore detected, more than once. Though three fiber optic cables are shown in FIG. 3G, one or more fiber optic cables are contemplated. When one fiber optic cable is used and there are multiple emission lights captured having different wavelengths, the different wavelengths cause the different emission lights to scatter at different angles. Therefore, a single fiber optic cable may be capable of transmitting multiple emission lights for detection.

Alternatively, each fiber optic cable receives emission lights having different wavelengths. The fiber optic cables may be preceded by a prism, a grating, or the like, to disperse the signal into signal components across the fiber optic cables. Each fiber optic cable may receive a different component and transmit the component accordingly. Alternatively, each fiber optic cable may be preceded by a respective filter, such that each filter permits passage, and subsequent detection, of a specific wavelength, and whereby no two filters permit passage of overlapping or equal wavelengths. The fiber optic cables then transmit the emission lights to the detector 374 or many detectors. When many detectors are used, each fiber optic cable transmits the emission light to a respective detector. The fiber optic cables may be formed in any appropriate configuration, such a ring, in a line, in a stack, staggered, or the like, such that each fiber optic cable is capable of receiving emission light.

### II. Imaging

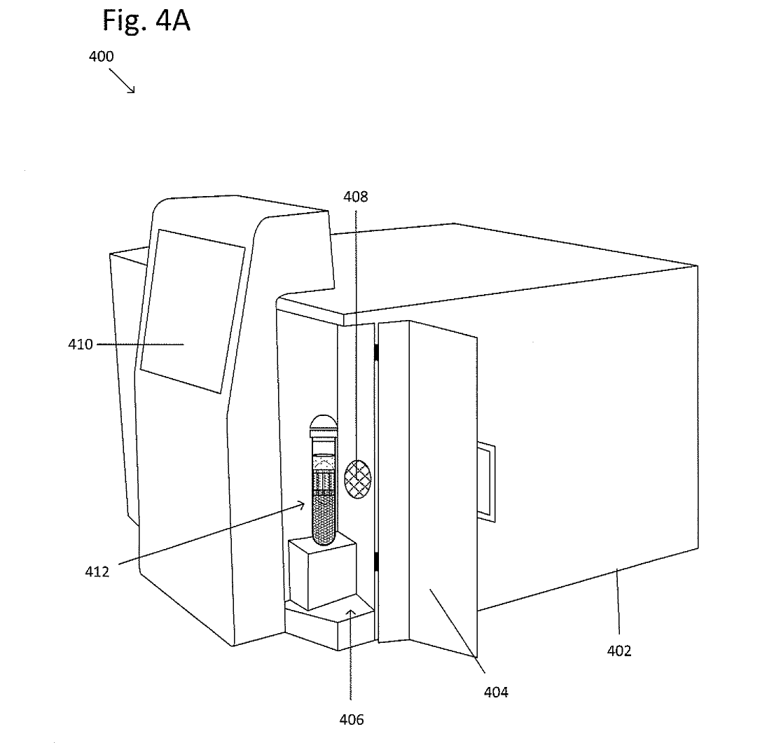

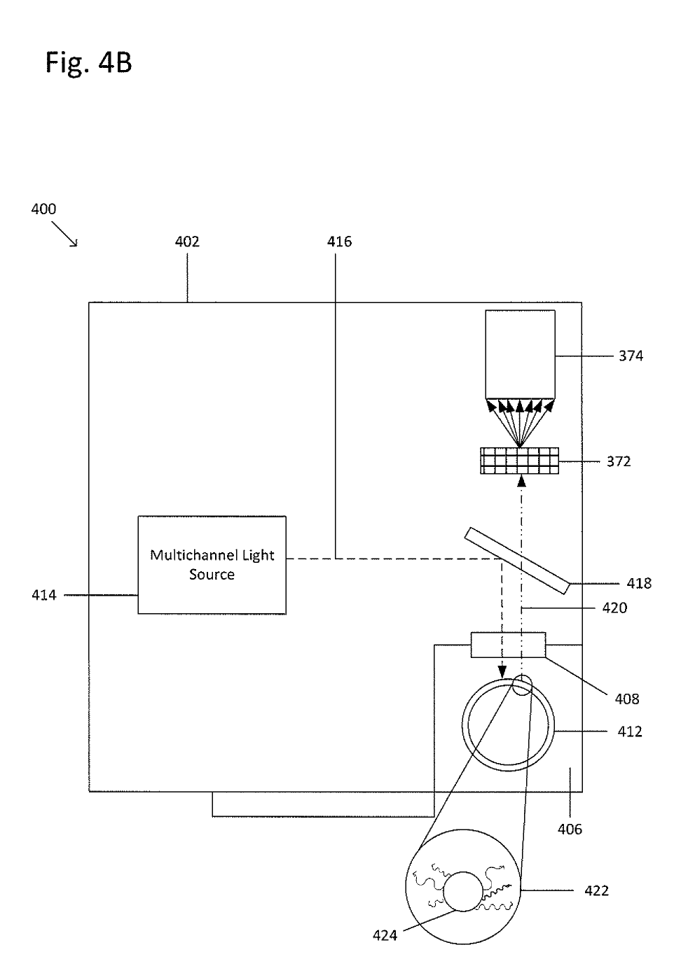

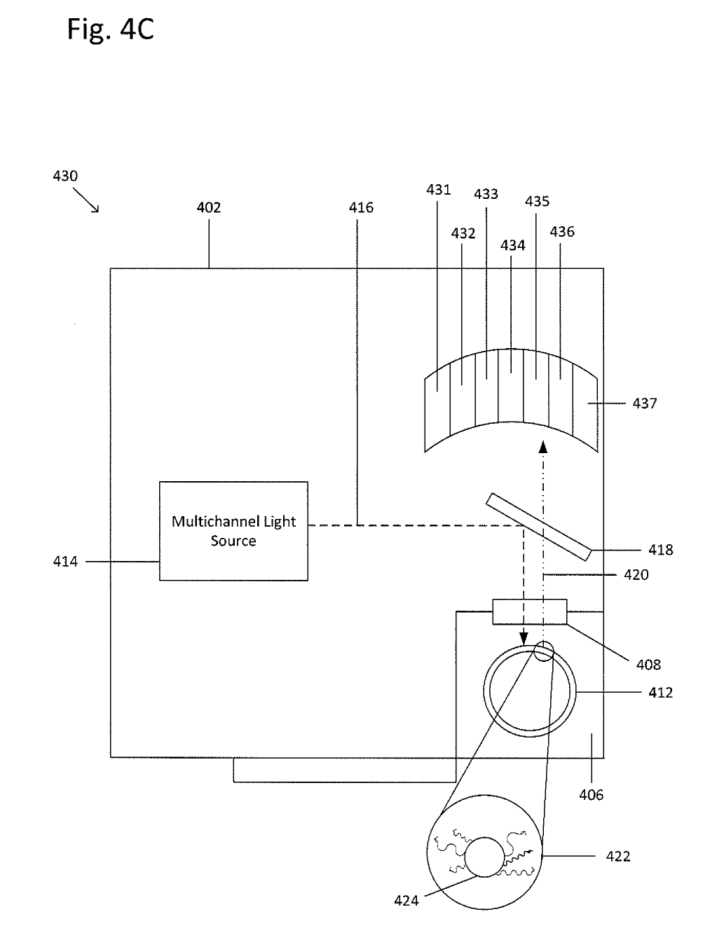

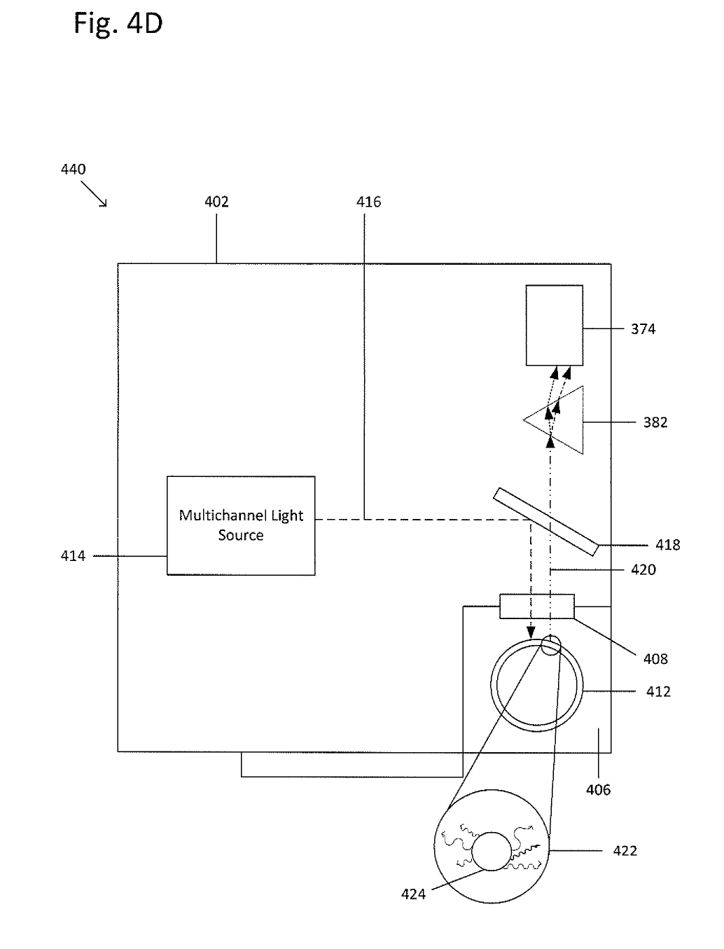

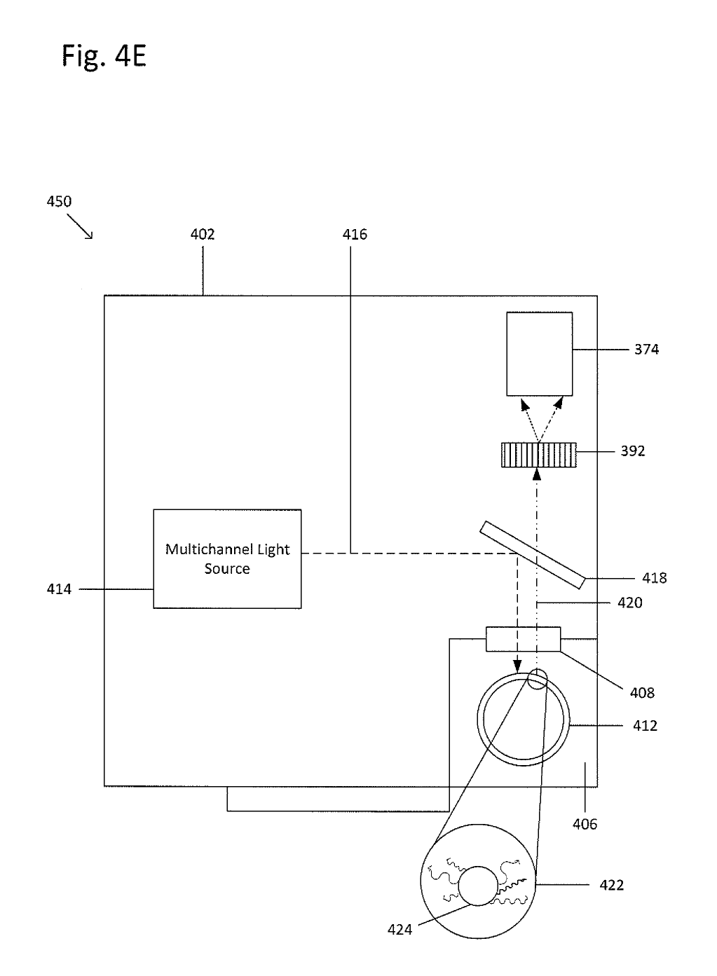

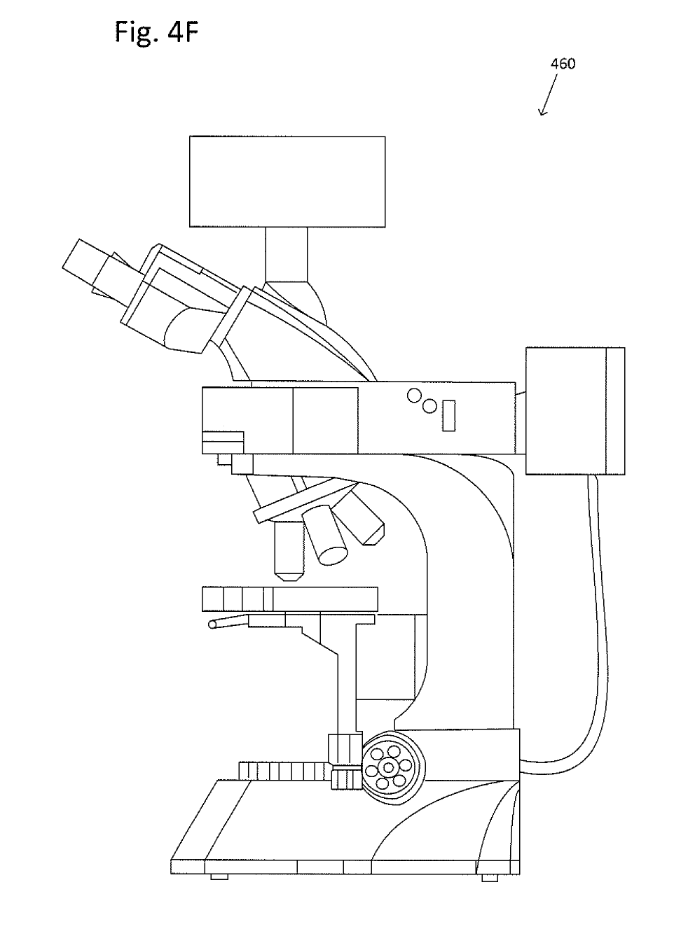

FIG. 4A shows an imager 400. The imager 400 includes a housing 402, a scanning compartment 406, a door 404, and an objective 408. The imager 400 also includes a multichannel light source (not shown) and a detector (not shown). The imager 400 may also include a screen 410 to display results, input imaging parameters, or the like. The imager 400 images a vessel 412 to detect and characterize a particle, such as a target analyte, of a fluid, a solution, a suspension, or the like. The vessel 412 may be a tube or a slide. The imaging techniques, such as multiple z stacks or optical axis integration, allows for imaging of the particle in a multitude of focal planes. The scanning compartment 406 is a chamber into which the vessel 412 is placed for imaging. The scanning compartment 406 may include bearings and chuck to rotate and vertically translate the vessel 412 within the scanning compartment 406 so as to image all areas of the vessel 412. The chuck may be rotated by a motor or driver (not shown) within the imager 400. The door 404 closes the scanning compartment 406 and prevents outside light from entering the scanning compartment 406 during the imaging process. The objective 408 collects and focuses excitation light from the multichannel light source (not shown) on the vessel 412. When the particle within the vessel 412 is excited and produces an emission light, the objective 408 collects the emission light and focuses the emission light on a singular point, such as the detector (not shown). Alternatively, the scanning compartment 406 may be or include a platform for holding the vessel 412 substantially level.

FIG. 4B shows a top down cut away view of the imager 400. The imager 400 further includes the multichannel light source 414, a polychroic mirror 418, the detector 374, and the photomask 372. The multichannel light source 414 emits an excitation light 416 which is reflected off of the polychroic mirror 418, through the objective 408, and into the vessel 412. The particle 424, as shown in snapshot 422, being excited by the excitation light 416, produces an emission light 420. The emission light 420 is collected by the objective 408, passes through the polychroic filter 418, and to the photomask 372 and detector 374 for analysis. The photomask 372 causes a singular signal to appear multiple times on the detector 374 in an array of X×Y, where X and Y are integers greater than or equal to 1. The photomask 372 may include a lens array of A×B, such that A and B are integers greater than or equal to 1, to project each copied image onto the detector 374. A filter array (not shown) may be used intermediate of the photomask 372 and the detector 374. The filter array (not shown) includes M×N filters, where M and N are integers greater than or equal to 1 and where there are an equal number of filters in the filter array and images produced by the photomask 372. Each filter of the filter array (not shown) may be a narrow bandpass filter. Each filter of the filter array (not shown) corresponds to an image produced by the photomask 372. The filter array (not shown) is configured to separately analyze each copied image for a wavelength specific to the individual copied image. The filter array (not shown) may further be configured such that none of the filter ranges overlap with another filter range (i.e. no two filters overlap in wavelength spectra permitted to pass through). The detector 374, based on the number of copies produced by the photomask 372, is configured to capture the signals each individual image. For example, a photomask produces a 5×5 array, thereby resulting in 25 images. A first image is analyzed for a 480 nm wavelength; a second image is analyzed for a 560 nm wavelength; and so on. The detector 374 is therefore capable of analyzing and detecting multiple wavelengths using a single image, though copied multiple times. The detector 374 may be a single detector or a detector array, such that each detector of the detector array receives a corresponding signal and is configured to only detect a specific wavelength range, such that no two wavelength ranges from different detectors in the detector array overlap. The detector array (not shown) includes O×P filters, where O and P are integers greater than or equal to 1.

Alternatively, a tunable filter or a filter wheel including multiple bandpass filters may be used. Multiple images may be taken, whereby each image only contains a singular wavelength, and each image is then analyzed individually. For example, a first bandpass filter on the filter wheel may allow light having a wavelength between 475 nm and 485 nm to pass. The 480 nm wavelength will pass and an image will be taken. The bandpass filter, being either tunable or having a multiple filters, each filter having a distinct range, may then permit the passage of light having a wavelength of 550 nm to 560 nm. An image may then be taken. Each image, having distinct wavelengths displayed, may be analyzed. A secondary filter (not shown) may also be used to permit the passage of emission light or lights, while inhibiting the passage of scatter from the light source 414.

FIG. 4C shows a top down cut away view of an imager 430. The imager 430 is similar to the imager 400 discussed above, except that imager 430 includes detectors 431-437, such that each detector is capable of detecting a specific range of wavelengths or an individual wavelength that does not overlap with any other wavelength or wavelengths detectable by another detector. The detectors 431-437 may be formed in any appropriate configuration, such a ring, in a line, in a stack, staggered, or the like, such that each detector is capable of receiving the emission light 420 or a filtered portion of the emission light 420. It should be further noted that any number of detectors equal to or greater than 2 are contemplated. The detectors 431-437 may include a respective filters, such as narrow bandpass filters or tunable filters, to permit passage, and subsequent detection, of a specific wavelength. The number of detectors is only limited by the number of distinct quantum dots being used. For example, a system which uses only 5 MDot ensembles only requires 5 detectors, whereas a system that uses 100 MDot ensembles requires 100 detectors.

Each one of the detectors 431-437 is configured to detect a single emission wavelength, such that none of the detector ranges overlap with another detector range. For example, an analyte may be conjugated with quantum dots from different MDot ensembles. A first detector detects a first wavelength emission; a second detector detects a second wavelength emission; and so on.

FIG. 4D shows an imager 440. The imager 440 is similar to the imager 400 discussed above, except that the imager 440 includes the prism 382. The prism 382 is an object which refracts light. As light enters a new medium, the light changes speed, thereby refracting the light, which enters the medium a different angle. The individual light colors making up the light refract are refracted differently, thereby leaving the prism 382 at different angles. By placing the prism 382 in front of the detector 374, the components of the original signal can be dispersed across the detector 374 due to the different angles at which the components are refracted by the prism 382. Breaking up the original signal into its components allows for the detection of the individual components, as the components do not mix with, dilute, weaken, or attenuate each other. The imager 440 may also include a second objective (not shown). The second objective (not shown) collects all of the emission lights and focuses the emission lights on a singular point, such as on the prism 382. Furthermore, many prisms, such as two or more, may be used in succession so to cause greater dispersion over the detector 374, as each successive prism increases the dispersion of the signal.

FIG. 4E shows an imager 450. The imager 450 is similar to the imager 440 discussed above, except that the imager 450 includes the grating 392. The grating 392, such as a diffraction grating, is an object which splits and diffracts light into individual wavelength components. Similar to the prism 382 discussed above, by placing the grating 392 in front of the detector 374, the components of the original signal can be dispersed across the detector 374 due to the different angles at which the components are split and diffracted by the grating 392. Breaking up the original signal into its components allows for the detection of the individual components, as the components do not mix with, dilute, weaken, or attenuate each other. The imager 450 may also include a second objective (not shown). The second objective (not shown) collects all of the emission lights and focuses the emission lights on a singular point, such as on the grating 392. Furthermore, many gratings, such as two or more, may be used in succession so to cause greater dispersion over the detector 374, as each successive grating increases the dispersion of the signal.

Alternatively, the grating prism may be used. The grating prism is a combination of a prism and a grating, such that a chosen wavelength is permitted to pass straight through. The grating prism may act like a filter by passing a chosen wavelength. However, the grating prism does not reject other wavelengths, if present. By diffracting and/or refracting the non-desired wavelengths, the grating prism may allow for subsequent processing of the non-desired wavelengths.

An imager, similar to one shown in FIG. 4D, may include many fiber optic cables. Each fiber optic cable receives emission lights having different wavelengths. The fiber optic cables may be preceded by a prism, a grating, or the like, to disperse the signal into signal components across the fiber optic cables. Each fiber optic cable may receive a different component and transmit the component accordingly. Alternatively, each fiber optic cable may be preceded by a respective filter, such that each filter permits passage, and subsequent detection, of a specific wavelength, and whereby no two filters permit passage of overlapping or equal wavelengths. The fiber optic cables then transmit the emission lights to the detector 362 or many detectors. When many detectors are used, each fiber optic cable transmits the emission light to a respective detector. The fiber optic cables may be formed in any appropriate configuration, such a ring, in a line, in a stack, staggered, or the like, such that each fiber optic cable is capable of receiving emission light. The imager may include two or more fiber optic cables.

FIG. 4F shows an imager 460. The imager 460 may be a fluorescent microscope or a confocal laser scanning microscope for detection of up to 30 or more wavelengths in parallel. The imager 460 may be automated or manual. The imager 460 may image microscope slides. The imager 460 may include detection components (i.e. filters, optical regulators, detectors, etc.) similar to those shown in FIGS. 4B-4E.

It should be noted that the emission light 420, though represented by a single dashed line, may actually be composed multiple wavelengths. The emission light 420 was shown as the single dashed line to show the path taken by the emission light 420. The imager may be a fluorescent microscope, such as a stationary microscope and a scanning microscope.

The detector may include, but is not limited to, a charge coupled device (“CCD”), an active-pixel sensor, a CMOS sensor, a photodiode, and a photomultiplier tube. The signals captured by the detector or detectors may be integrated or compiled.

### III. Uses

After a quantum dot aggregate has been sorted into MDot ensembles, the MDot ensembles may be used in variety of applications, including, but not limited to, multiplexing a target analyte, tagging DNA origami, or within an illuminator.

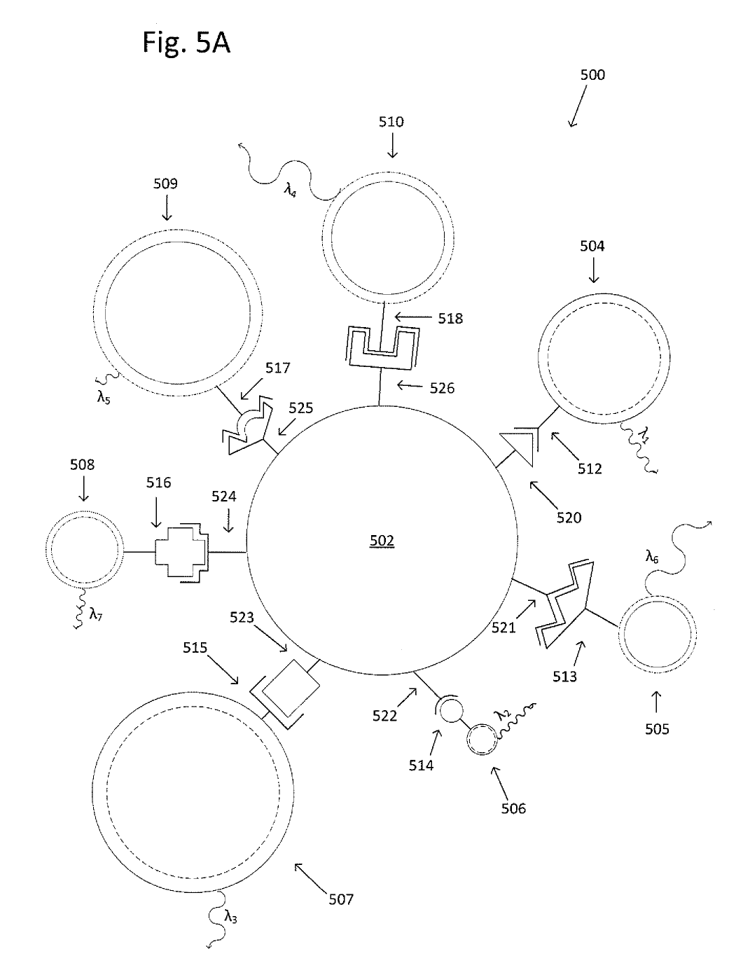

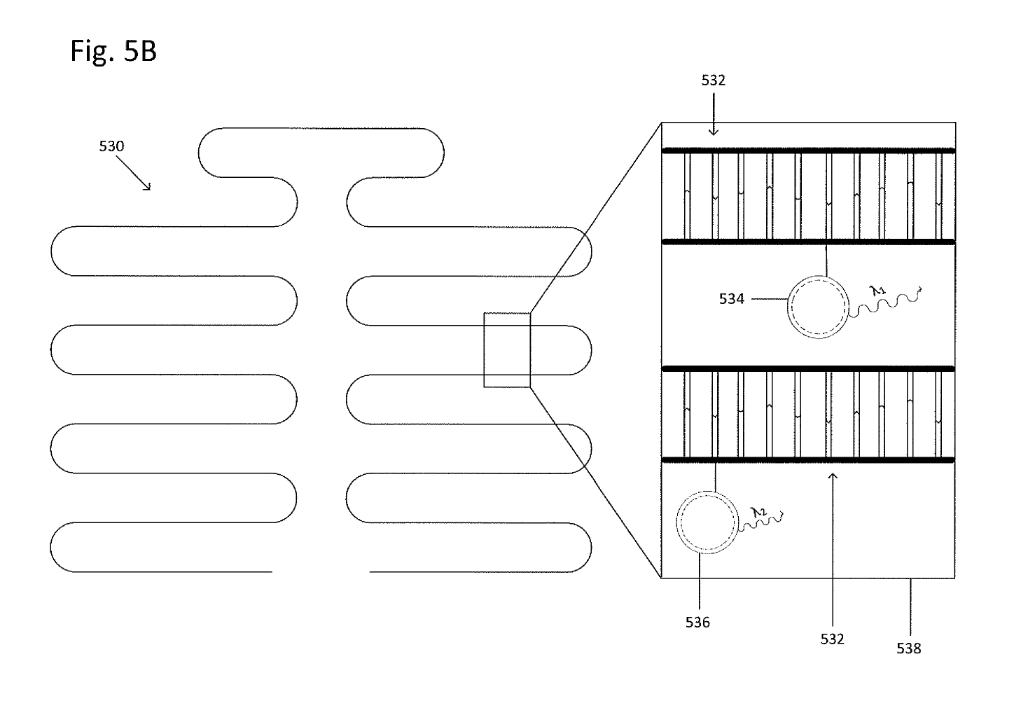

FIG. 5A shows a target analyte 502 which is bound by different ligands attached to quantum dots 504-510 to provide a multiplexed signal to characterize the target analyte 502. The target analyte 502 may include one or more biomarkers 520-526, based on nucleic acid sequences or expression levels, lipids, carbohydrates, or proteins. The biomarkers 520-526 may be on the surface of the target analyte 502, may be within the target analyte 502, or combinations thereof. Each one of the quantum dots 504-510 may be bound to a ligand 512-518. When excited by an excitation light from a light source (not shown), each one of the quantum dots 504-510 emits an emission light having different emission wavelengths λ₁-λ₇. The quantum dots of a MDot ensemble are bound to a ligand to attach to a specific marker. When the ligand is tagged by the quantum dots, the ligand is added to a sample which is suspected of including the target analyte 502. The target analyte 502 containing the appropriate biomarkers interact with the ligands 512-518 with the attached respective quantum dots, thereby forming a target analyte-quantum dot complex. The target analyte-quantum dot complex may be analyzed using techniques such as imaging, fluorescence activated cell sorting (“FACS”), or spectroscopy.

It may be desirous to use as many markers as possible to identify various characteristics of a target analyte. The target analyte may have a number of different types of surface markers. Each type of surface marker is a molecule, such an antigen, capable of attaching a particular ligand, such as an antibody. As a result, ligands can be used to classify the target analyte and determine the specific type of target analytes present in the sample by conjugating ligands that attach to particular surface markers with a particular quantum dot. For example, each type of quantum dot emits light in a narrow wavelength range of the electromagnetic spectrum called a “channel” when an appropriate stimulus, such as light with a shorter wavelength, is applied. A first type of quantum dot that emits light at a first wavelength can be attached to a first ligand that binds specifically to a first type of protein, while a second type of quantum dot that emits light at second wavelength can be attached to a second ligand that binds specifically to a second type of protein, and so on. The spectral purity of quantum dots permits 5 or more colors or emission ranges to be used, thereby permitting multiple ligands to be used and for multiple cellular occurrences and variations to be detected in the same sample. The channel color observed as a result of stimulating the target analyte identifies the type of protein, and because proteins can be unique to particular target analyte, the channel color can also be used to identify the target analyte.

Quantum dots also enable new applications such as improved illumination systems for microscopy, deterministic detection of rare or low level events, and significant increases in the number of reporting markers. Quantum dots may also be used to tag components of DNA, such as nucleotides, within DNA origami. This allows for the use of multiple quantum dots for fluorescent in-situ hybridization (“FISH”). Currently, implementation of quantum dot aggregates within DNA origami is limited due to the spectral overlap of the quantum dot aggregates. FIG. 5B shows tagged DNA origami 530. The DNA origami 530 includes a nucleic acid strand 532, as shown in snapshot 538 tagged with MDot ensembles 534 and 536. Nucleic acid origami, such as DNA origami, is the folding of a nucleic acid strand into various two- and three-dimensional shapes on a nanoscale level. The nucleic acid strand 532 forms from building blocks, such as nucleic acid strand components. Tagging various nucleic acid strand components, such as individual nucleotides or nucleotide sequences, with MDot ensembles 534 and 536 may permit multiplexing of a particular object, cell, or the like within a singular pixel or group of pixels on an imaging device. For example, using 10 different MDot ensembles within DNA origami may allow for the formation of many different combinations. The DNA origami may be tagged with the MDot ensembles 534 and 536 by using click chemistry or any other appropriate tagging method.

Alternatively, nucleic acid components, such as nucleotides or nucleotide sequences, or nucleic anomalies, such as single nucleotide polymorphisms, may be detected with a quantum dot from different MDot ensembles, thereby associating a specific wavelength or wavelength range with a specific nucleotide or nucleotide sequence. Specifically detecting each nucleic acid strand component allows for enumeration and detection of the various nucleic acid strand components. The nucleic acid strand may include a DNA strand, a RNA strand, or the like.

It should be understood that the method and system described and discussed herein may be used with any appropriate suspension or biological sample, such as blood, bone marrow, cystic fluid, ascites fluid, stool, semen, cerebrospinal fluid, nipple aspirate fluid, saliva, amniotic fluid, vaginal secretions, mucus membrane secretions, aqueous humor, vitreous humor, vomit, and any other physiological fluid or semi-solid. It should also be understood that a target analyte can be a cell, such as ova or a circulating tumor cell (“CTC”), a circulating endothelial cell, a vesicle, a liposome, a protein, a nucleic acid, a biological molecule, a naturally occurring or artificially prepared microscopic unit having an enclosed membrane, parasites, microorganisms, or inflammatory cells.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

I/We claim:

1. A monochromatic nanoparticle ensemble, comprising:
a plurality of fluorescent nanoparticles,
wherein each fluorescent nanoparticle of the plurality of fluorescent nanoparticles emits an emission light when excited by a stimulus, and,
wherein the emission light from each fluorescent nanoparticle deviates from a specified wavelength by a pre-determined amount.

2. The ensemble of claim 1, wherein the emission light from each fluorescent nanoparticle deviates from the specified wavelength by no more than approximately 10 nm.

3. The ensemble of claim 1, wherein the emission light from each fluorescent nanoparticle deviates from the specified wavelength by no more than approximately 5 nm.

4. The ensemble of claim 1, wherein the emission light from each fluorescent nanoparticle deviates from the specified wavelength by no more than approximately 1 nm.

5. The ensemble of claim 1, wherein the emission light from each fluorescent nanoparticle deviates from the specified wavelength by no more than approximately 0 nm.

6. The ensemble of claim 1, wherein the fluorescent nanoparticles are maintained at or below freezing temperature.

7. The ensemble of claim 1, wherein the fluorescent nanoparticles are maintained between freezing and room temperature.

8. The ensemble of claim 1, wherein the fluorescent nanoparticles are maintained at approximately room temperature.

9. The ensemble of claim 1, wherein the fluorescent nanoparticles are maintained above room temperature.

10. A method for sorting a fluorescent nanoparticle aggregate into at least one monochromatic particle ensemble, comprising:
providing a fluorescent nanoparticle aggregate in a solution, the fluorescent nanoparticle aggregate comprising a plurality of fluorescent nanoparticles;
exciting a fluorescent nanoparticle of the fluorescent nanoparticle aggregate with a stimulus to emit an emission light
detecting the emission light;
distributing the fluorescent nanoparticle into a container, wherein each fluorescent nanoparticle within the container deviates from a specified wavelength by a pre-determined amount.

11. The method of claim 10, wherein the pre-determined deviation is equal to or less than approximately 50 nm.

12. The method of claim 10, wherein the pre-determined deviation is equal to or less than approximately 10 nm.

13. The method of claim 10, wherein the pre-determined deviation is equal to or less than approximately 5 nm.

14. The method of claim 10, wherein the pre-determined deviation is equal to or less than approximately 1 nm.

15. The method of claim 10, wherein the pre-determined deviation is equal to approximately 0 nm.

16. The method of claim 10, wherein the fluorescent nanoparticles are excited and the emission light is detected at or below freezing temperature.

17. The method of claim 10, wherein the fluorescent nanoparticles are excited and the emission light is detected between freezing and room temperature.

18. The method of claim 10, wherein the fluorescent nanoparticles are excited and the emission light is detected at approximately room temperature.

19. The method of claim 10, wherein the fluorescent nanoparticles are excited and the emission light is detected above room temperature.

20. A monochromatic nanoparticle ensemble comprising:
a plurality of fluorescent nanoparticles, each fluorescent nanoparticle of the plurality of fluorescent nanoparticles emitting an emission light when excited by a stimulus, and,
a monochromatic nanoparticle ensemble emission spectrum that is less than or equal to one-half of a native aggregate emission spectrum,
wherein the native aggregate emission spectrum is the difference between the longest and shortest wavelengths within a native aggregate, and
wherein the monochromatic nanoparticle ensemble emission spectrum is the difference between the longest and shortest wavelengths of the plurality of fluorescent nanoparticles.
